(19) United States
(12) Patent Application Publication
(10) Pub. No.: US 2021/0259919 A1
(43) Pub. Date: Aug. 26, 2021

(54) Hemorrhoid mitigation device

(71) Applicant: David Salem Magarian, Concord, CA (US)

(72) Inventor: David Salem Magarian, Concord, CA (US)

(21) Appl. No.: 16/796,778

(22) Filed: Feb. 20, 2020

(51) Int. Cl. A61H 39/04; A61N 2/00

(52) U.S. Cl. CPC A61H 39/04; A61N 2/004; A61H 2039/005; A61H 2201/1654; A61H 2205/06

(57) ABSTRACT

A wearable hemorrhoid relief device which provides stimulation to the “Ex-UE-2 Two Whites ERBAI” acupressure points on the forearm of a user. Various mounting structures may be used to hold two raised contact buttons in place over the “Ex-UE-2 Two Whites ERBAI” acupressure points. By providing physical and/or magnetic stimulation of the “Ex-UE-2 Two Whites ERBAI” acupressure points in a wearable device, a user is able to relieve hemorrhoid symptoms at their own discretion.

### BACKGROUND OF THE INVENTION

Irritation and pain caused by hemorrhoids is a serious issue affecting millions of people. Hemorrhoids are sometimes addressed surgically to remove the offending tissue or assuaged through the use of topical medication, prevented through dietary modification, and treated through acupuncture using a variety of techniques including stimulation of the two “Ex-UE-2 Two Whites ERBAI” acupressure points used in Traditional Chinese Medicine (TCM) to provide hemorrhoid relief. While acupuncture and acupressure can be helpful, the treatment has been limited typically to a weekly or twice weekly visit to an acupuncture office.

This invention provides a means to provide stimulation of the two “Ex-UE-2 Two Whites ERBAI” acupressure points outside of a clinical setting. By doing so a user is enabled to relieve hemorrhoid symptoms at their own discretion.

### FIELD OF THE INVENTION

The invention pertains to a device for stimulating a pair of proximal acupuncture or acupressure points on the human forearm. More specifically, the present invention relates to an acupressure device for relieving hemorrhoid symptoms.

### DESCRIPTION OF RELATED ART

The effectiveness of acupuncture and acupressure for relieving pain and for treating certain physical disorders has been known for several thousand years. In acupuncture, fine needles are inserted into the skin at specific locations on the anatomy in order to treat specific disorders. Since it is an invasive procedure, acupuncture should be administered only by a skilled practitioner. Even with normal clinical precautions, there is a danger of infection at acupuncture sites.

Due, at least in part, to the disadvantages of acupuncture, acupressure has enjoyed increasing popularity. The same anatomical locations used for acupuncture are also used for acupressure; however, only pressure is applied to the location. Pressure alone has proven to be effective for the treatment of certain disorders and for certain symptomatic relief. Other methods of stimulation for acupuncture points including magnetic stimulation (achieved by placing a magnet on the specified acupuncture point) and electrical stimulation (achieved by running electrical charge through acupuncture needles or contact patches applied to the skin) have also been found to be effective.

Various devices have been developed for applying acupressure to the human body. For example, U.S. Pat. No. 4,479,495 to Isaacson and U.S. Pat. No. 4,716,898 to Chauve, et al. show acupressure devices in the form of bands that may be applied around an extremity.

The closest know prior art is a nausea control device for suppressing nausea by stimulation of the pericardium six (P6) acupuncture point at a human wrist. For example, U.S. Pat. No. 4,981,146A to Bertolucci, et al. shows a device that includes electronic circuitry for imparting electrical impulses via positive and negative electrodes to the P6 acupuncture point for alleviating nausea. No prior art was found for devices specifically intended to stimulate the Ex-UE-2 Two Whites ERBAI points to relieve hemorrhoid symptoms.

### SUMMARY OF THE INVENTION

The device is a hemorrhoid mitigation devise which provides physical and/or magnetic stimulation to the two “Ex-UE-2 Two Whites ERBAI” acupressure points used in Traditional Chinese Medicine (TCM) to provide hemorrhoid relief. The device is composed of a textile band or strap and two raised contact buttons disposed in the textile in position over the two “Ex-UE-2 Two Whites ERBAI” acupressure points on a user's forearm.

### DRAWINGS—FIGURES

FIG. 1 shows a perspective view of the device composed of a textile band or strap and two raised contact buttons secured by means of a hook and loop fastening system

FIG. 2 shows a perspective view of the device composed of a tubular textile band or strap and two raised contact buttons

FIG. 3 shows a profile view of one contact button attached by means of a push rivet

FIG. 4 shows an exploded cross-section view of one contact button assembly including a molded hemispherical contact shape, a ring magnet disposed inside the contact shape and a push rivet

FIG. 5 shows a perspective view of component parts of one contact button including a molded hemispherical contact shape, a ring magnet, and a push rivet

FIG. 6 shows the location of the “Ex-UE-2 Two Whites ERBAI” acupressure points on the palmer aspect of a human forearm

FIG. 7 shows the device worn on a human forearm

### DRAWINGS—REFERENCE NUMERALS

101 raised contact buttons
102 textile band or strap
103 hook and loop fastening system
201 tubular textile band or strap
301 molded hemispherical shape
302 push rivet fastener
401 embedded countersunk ring magnet
601 cubital crease
602 palmar side of the forearm
603 “Ex-UE-2 Two Whites ERBAI” acupressure points
604 flexor carpi radialis tendon
605 reference line use to locate “Ex-UE-2 Two Whites ERBAI” points
606 wrist crease
607 palm side of hand

### DETAILED DESCRIPTION

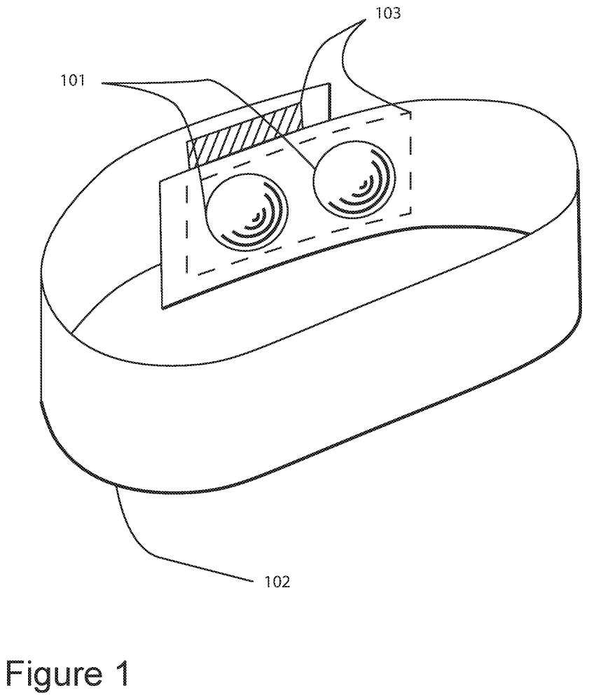

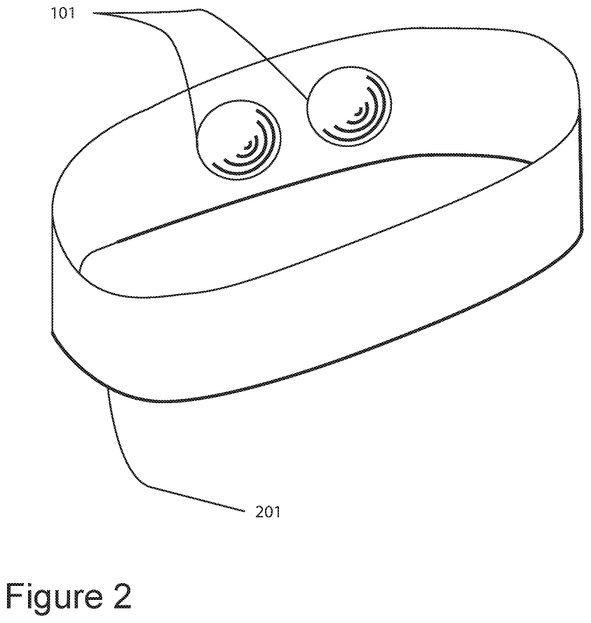

FIG. 1 shows a perspective view of the device composed of a textile band or strap 102 and two raised contact buttons 101 extending from textile positioned over the “Ex-UE-2 Two Whites ERBAI” acupressure points on a user's forearm. The textile band shown in FIG. 1 includes a hook and loop fastening system 103. Adjustability of the textile band or strap is achieved through any combination of elastic or non-elastic materials, hook and loop fasteners, snaps, and or buckles. FIG. 2 shows a perspective view of an embodiment where a fastening system is inherent in the structure of a tubular textile band or strap 201. The textile band or strap may be elastic in nature or non-stretching in nature.

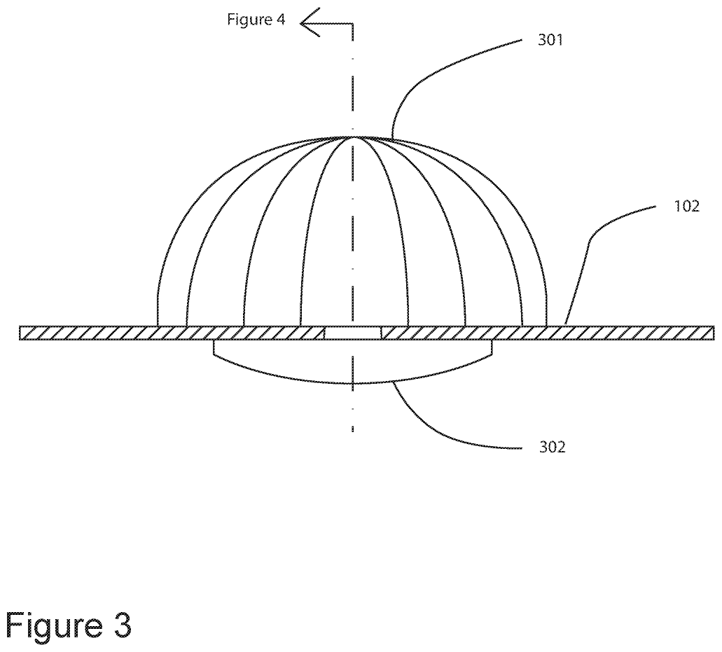

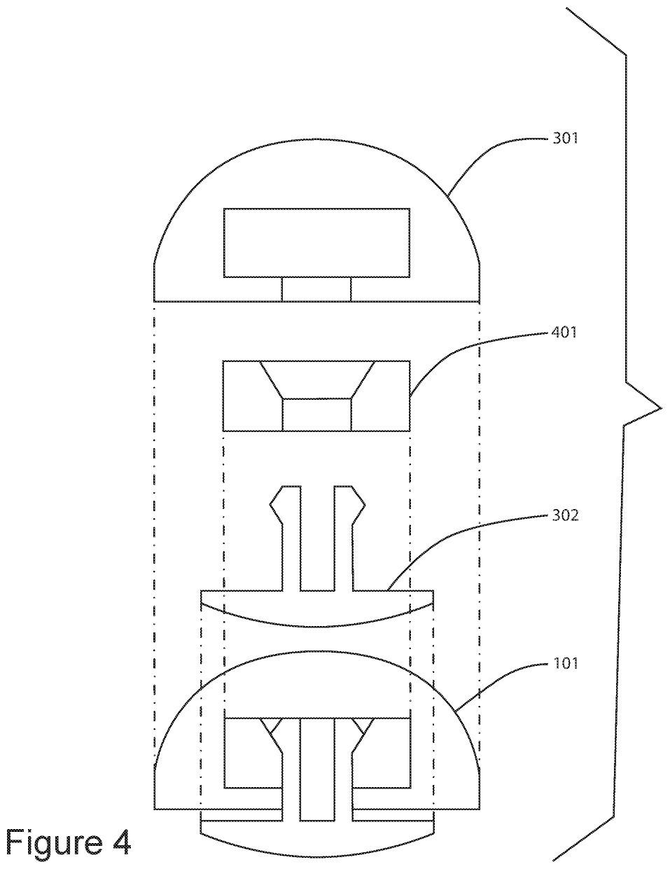

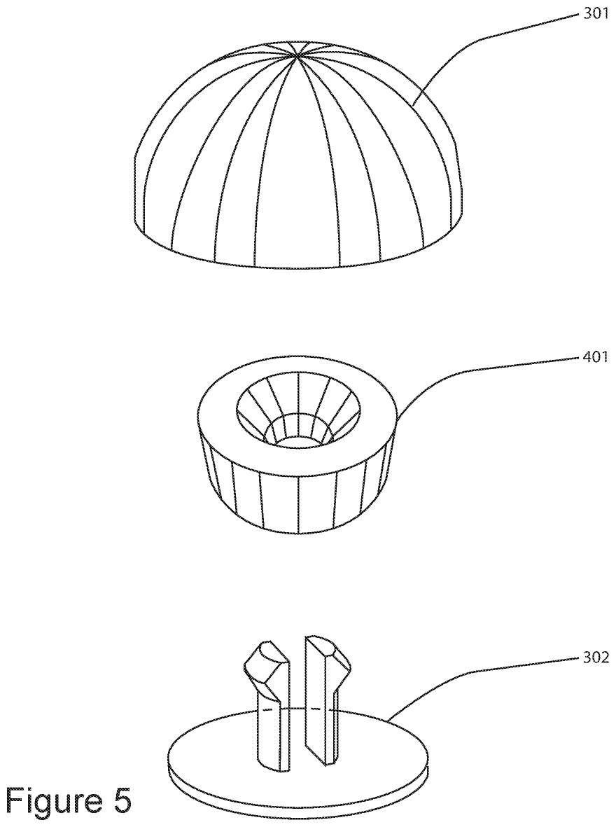

As shown in FIG. 3 the raised contact buttons 101 are composed of a molded hemispherical shape 301 and a snap fastener 302 used to fix the molded hemispherical shape 301 in place on the textile 102. Magnetic stimulation at the of the “Ex-UE-2 Two Whites ERBAI” acupressure points 603 can be achieved as seen in FIGS. 4 and 5 by embedding a magnet 401 in the molded hemispherical shape 301. In this embodiment the magnet 401 embedded in the molded hemispherical shape 301 is a countersunk ring magnet through which the push rivet fastener 302 is inserted. There are many potential embodiments for the fastener used to dispose the raised contact buttons in the textile band, such as pop rivets, push rivets, snaps, and glues. Materials used to construct the contact buttons include but are not limited to plastics, silicones, rubbers, metals, or clays.

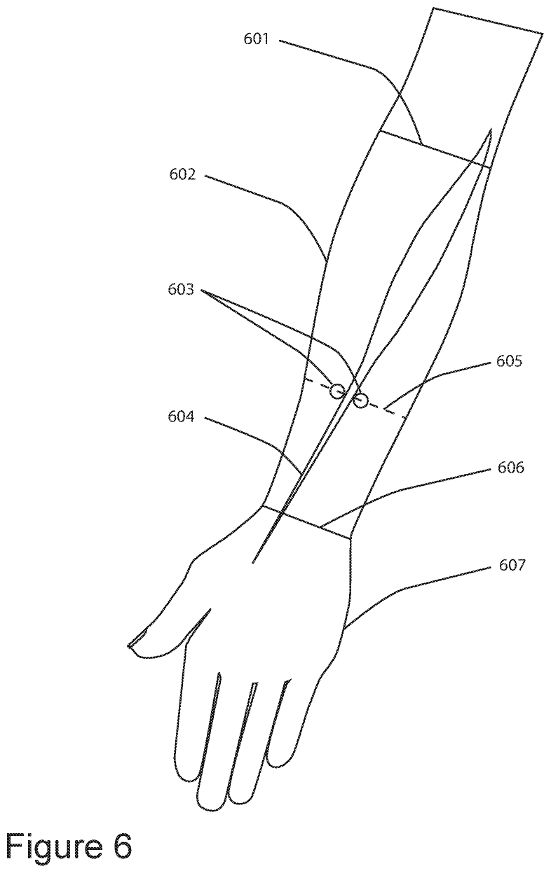

FIG. 6 shows the location of the “Ex-UE-2 Two Whites ERBAI” acupressure points 603, a pair of points located on the palmar side of the forearm 602 one third of the distance up the forearm at a reference line 605 measured from the wrist crease 606 to the cubital crease 601 on both sides of the flexor carpi radialis tendon 604.

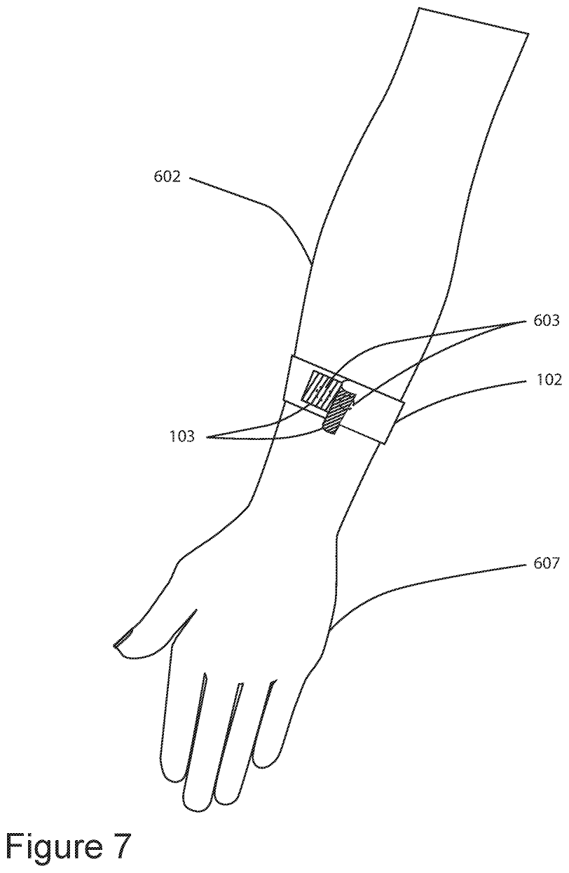

FIG. 7 shows the device worn on a human wrist such that the textile band or strap 102 adjusted with hook and loop fasteners 103 secures the two raised contact buttons 101 in contact with the skin at the “Ex-UE-2 Two Whites ERBAI” acupressure points 603. A user operates the device is by wearing the device as shown in FIG. 7.

Thus, the reader will see that any of the constructions of the Hemorrhoid mitigation device discussed above enable a user to relieve hemorrhoid symptoms at their own discretion.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A hemorrhoid mitigation device for stimulation of the “Ex-UE-2 Two Whites ERBAI” acupressure points comprising:
a) a textile surface configured to wrap around a human forearm, that is either continuous or having a first end and a second end having a hook and loop fastening system, a buckle system, or other mechanism to attach the two ends
b) two raised contact buttons disposed in a portion of the textile to contact the skin providing physical and/or magnetic stimulation of the “Ex-UE-2 Two Whites ERBAI” acupressure points on a human forear
